(19) United States
(12) Patent Application Publication
(10) Pub. No.: US 2007/0027446 A1
(43) Pub. Date: Feb. 1, 2007

(54) Method of removing a tattoo

(75) Inventor: Nigel M. Goble, Berkshire (GB)

Correspondence Address: OLIFF & BERRIDGE, PLC, P.O. BOX 19928, ALEXANDRIA, VA, 22320, US

(73) Assignee: RHYTEC LIMITED, Hungerford (GB)

(21) Appl. No.: 11/491,999

(22) Filed: Jul. 25, 2006

Related U.S. Application Data
(63) Continuation-in-part of application No. 10/792,765, filed on Mar. 5, 2004.
Application No. 10/792,765 is a continuation-in-part of application No. 09/789,550, filed on Feb. 22, 2001, now Pat. No. 6,723,091.
(60) Provisional application No. 60/183,785, filed on Feb. 22, 2000.

(30) Foreign Application Priority Data
Jun. 30, 2006 (GB) 0613134.6

(51) Int. Cl. A61B 18/04; A61F 7/00; A61F 7/12

(52) U.S. Cl. 606/28; 607/96

(57) ABSTRACT

A cosmetic method of removing a tattoo from skin tissue is disclosed. The method uses a source of thermal energy with a low thermal time constant, and comprises the step of operating the thermal energy source to form first and second adjacent regions of thermally-modified tissue. The first region overlies the second region and is thermally modified to a greater extend than the second region. The method is such that tattoo pigment(s) contained in the first region are transepidermically eliminated, and tattoo pigment(s) in the second region are removed by an inflammatory response.

This application is a Continuation-in-Part of U.S. patent application Ser. No. 10/792,765, filed Mar. 5, 2004 that is a Continuation-in-Part Application of U.S. patent application Ser. No. 09/789,500, filed Feb. 22, 2001, that in turn claims the benefit of priority of U.S. Provisional Patent Application No. 60/183,785, filed Feb. 22, 2000. The complete disclosures of U.S. Provisional Patent Application No. 60/653,498, U.S. patent application Ser. No. 10/792,765, U.S. patent application Ser. No. 09/789,500, and U.S. Provisional Patent Application No. 60/183,785, including the specifications, drawings, and claims are incorporated herein by reference in their entirety.

This invention relates to a cosmetic method of removing a tattoo, and in particular tattoo inks irrespective of colour, in combination with a regeneration of the reticular architecture of the dermis.

Human skin has two principal layers: the epidermis, which is the outer layer and typically has a thickness of around 120 μm in the region of the face, and the dermis which is typically 20-30 times thicker than the epidermis, and contains hair follicles, sebaceous glands, nerve endings and fine blood capillaries. By volume the dermis is made up predominantly of the protein collagen.

Tattoos applied to the skin have been performed for over thousands of years. The process is based on implanting inks or pigments (hereinafter referred to as “pigments”) into the skin using a sharp object to pierce the outer layer of the skin to drive the pigments to a level around, or just under, the dermo-epidermal junction of the skin (DEJ). Typically, the pigments will migrate deeper into the dermis as the tattoo matures.

For a variety of reasons, often driven by social stigma, as a person ages there comes a desire for tattoos to be removed. Many techniques to remove tattoos have been tried over the years, including chemical, mechanical, surgical and thermal techniques. All these techniques are associated with alterations in the natural skin pigment melanin, resulting in either too little (known as hypopigmentation) or in too much (known as hyperpigmantation), and a permanent scar.

With the advent of commercially-available lasers in the 1960s, the ability to target different tattoo ink colours with specific wavelengths of light improved the prospects of effective tattoo removal. The most popular form of laser for achieving this was, and still remains, the Q-switched ruby laser.

The pigments used for tattoos consist of insoluble, sub-micron particles that have become incorporated in cells of the DEJ and the dermis by a process of phagocytosis. These particles can be targeted by specific wavelengths of laser light which, when delivered as a series of pulses, produces a process known as selective photothermolysis. Inside the pigments, the light is converted to heat extremely quickly with temperatures exceeding 1000° C. The temperature rise is so rapid that it is accompanied by a photoacoustic shock which fragments the pigment particles and kills the cells into which they had been incorporated. The debris produced by the process is phagocytosed by inflammatory cells responding to the highly-localised injury, and pigments appear in lymph nodes draining the treated area. The remaining pigments become diffused within the dermis, such that they become less visible. Whist the bulk of the skin remains unaffected by the treatment, there is nonetheless some sloughing of the epidermis. As part of the sloughing, some pigments are truly eliminated from the body, so-called transepidermal elimination.

For a professional tattoo its takes ten to fifteen treatment sessions, and sometimes the use of additional laser wavelengths such as alexandrite (755 nm) or Nd:YAG (1064 or 532 nm) to complete the removal. The problem, however, is that the complex pigments used in modern inks, and applied in decorative tattoos, can both change colour and be resistant to laser treatment, simply because the laser wavelengths appropriate to some pigments do not exist. Resistive colours include yellow, green and blue, and the resistance increases when these are combined with titanium dioxide to brighten their colour. Those pigments containing titanium dioxide and ferric oxide may also undergo a chemical transformation induced by the photothermal reaction, resulting in a darkening of the tattoo. Flesh colours and reds used in permanent makeup are particularly prone to this phenomenon. Once darkened, removal using lasers becomes virtually impossible. One further problem occurs when the amount of pigment is very high, such that normal cells are also destroyed by the intensity of the photothermal effect, thereby inducing scar formation.

A common aim of many cosmetic procedures is to improve the appearance of a patient's skin. For example, a desirable clinical effect in the field of cosmetic procedures is to provide an improvement in the texture of ageing skin, and to give it a more youthful appearance. These effects can be achieved by the removal of a part or all of the epidermis, and on occasions part of the dermis, causing the growth of a new epidermis having the desired properties.

These methods are referred to as non-surgical techniques, as they are not associated with an incision or surgical manipulation of the tissue as occurs in, for example, a surgical face-lift where an incision is made through the skin, redundant skin is removed and, when the incision is closed, the skin is pulled taut. The effects of these non-surgical methods rely on the healing response of the skin to the superficial injury, so that they must not go “through the skin” or a scar would result as occurs with a surgical incision. The disadvantage of each of these methods is that the surface of the skin is effectively removed at the time of the procedure, and that the depth of effect is dependent on the depth of the skin removed at that time. There is little or no modification of tissues beneath the point of removal, so that it is the formation of scar tissue at the level of removal that provides the result.

Plasma Skin Regeneration (PSR) is a non-surgical technique employing an invention disclosed in U.S. patent application Ser. No. 10/792,765, filed 5 Mar. 2004, the disclosure of which (including the specification, drawings and claims) is incorporated by reference in its entirety. The method of treating the skin using PSR involves exposing the skin to millisecond pulses of nitrogen or other diatomic gas that has been ionised using ultra-high frequency radiofrequency energy. The ionised gas stores energy that is given up to the skin as thermal energy, producing a heating of both the epidermis and deeper dermis of the skin. The depth of the effect is a function of the power setting and the moisture content of the skin, provided the distance and angle of the plasma pulse remains constant with respect to the skin surface.

The energy locked up in the nitrogen gas takes the form of ionisation, splitting of the nitrogen molecules and oscillatory motions of the molecules. On impact with the skin, this energy is given up directly to the fluid content of the skin to vaporise at least part of the skin. As heat is given up to the skin as a whole, variations in water content will modify its bulk thermal characteristics. No intermediary is involved, as occurs with lasers that rely on a target chromophore for conversion of light energy to thermal energy. The effect is more uniform and less disruptive as a result. Consistent with this, the treatment of photodamage using lasers often involves more than one pass over the surface, with the treated skin being wiped away between passes. The wiping is necessary, not only to increase the depth of penetration, but also to refresh the chromophore.

The present invention provides a cosmetic method of removing a tattoo from skin tissue, the method comprising the step of operating a source of thermal energy with a low thermal time constant and directing it at the surface of the skin overlying a tattoo to be removed; forming first and second adjacent regions of thermally-modified tissue, said first region overlying said second region and being thermally modified to a greater extent than said second region; and causing tattoo pigment(s) contained in the first region to be transepidermally eliminated, and tattoo pigment(s) in the second region to be removed by an inflammatory response.

The invention also provides a cosmetic method of removing a tattoo from skin tissue using a source of thermal energy with a low thermal time constant, the method comprising the step of operating the thermal energy source and directing it at the surface of the skin overlying a tattoo to be removed; forming first and second adjacent regions respectively of thermally-damaged and thermally-modified tissue; and causing tattoo pigment(s) contained in the first region to be transepidermally eliminated, and tattoo pigment(s) in the second region to be removed by an inflammatory response.

In a preferred embodiment, the thermal energy source is operated for a singe pass over the skin surface, the thermal energy source being arranged to have an energy setting dependent on the desired depth of effect. Alternatively, the thermal energy source is operated over at least two passes over the skin surface, the energy levels of the passes being chosen dependent on the desired depth of effect.

In either case, the energy setting of the thermal energy source may be such as to create vacuolation on the first pass. In the latter case, the energy setting of the thermal energy source may be such as not to create vacuolation on the first pass, thereby enabling a second pass without removing the treated skin.

Preferably, the energy setting of the thermal energy source is such as to preserve the integrity of the epidermis as a biological dressing.

In a preferred embodiment, the thermal energy source is operated so that a line of cleavage occurs within the skin 2 to 5 days following treatment, the line of cleavage occurring between said first and second regions. In one particular case, the operation of the thermal energy source may be such as to form a line of cleavage from 2 to 3 cells deep.

Advantageously, the operation of the thermal energy source is such that the tissue in the first region is sloughed tissue. In this case, the sloughed tissue is removed once a new epidermis has been substantially generated in the region of the line of cleavage.

Preferably, the tissue below the line of cleavage in said second region includes the lower epidermis, the basal membrane and the DE Junction. More preferably, at least the thermally-modified basal membrane and the DE Junction are regenerated.

In one particular case, the line of cleavage forms below areas of retained tattoo pigment(s).

Preferably, the operation of the thermal energy source is such as to denature cellular elements containing tattoo pigment(s) in the second region.

In a preferred embodiment, the tissue in said second region undergoes a regenerative process following regeneration of the epidermis.

In this case, the reticular architecture of the dermis is regenerated in whole, or in part, by fibroblasts less exposed to the effects of UV radiation.

The collagen architecture and/or elastin architecture and/or the GAGS of the dermis is regenerated in whole, or in part, by fibroblasts less exposed to the effects of UV radiation.

Preferably, the healing process is such that risk of scarring and hypo pigmentation is substantially eliminated.

A further benefit of the method is that the pigment retained deeper in the dermis below the second region is brought closer to the surface of the skin following a single treatment. This deeper pigment may then be removed using a second treatment.

Another benefit of the treatment is that the regenerated skin exhibits more normal characteristics when compared to laser treatments, particularly as it applies to the translucency of the skin. Laser treatments may increase the translucency, and hence make more visible any pigments retained more deeply in the dermis. The current invention simulates the regeneration of more normal skin, such that the appearance of retained pigment becomes less obvious.

In a preferred embodiment, the source of thermal energy is an instrument having an electrode connected to a power output device, and wherein the power output device is operated to create an electric field in the region of the electrode; a flow of gas is directed through the electric field to generate, by virtue of the interaction of the electric field with the gas, a plasma; the plasma is directed onto the tissue for a predetermined period of time; and the power transferred into the plasma from the electric field is controlled so as to desiccate at least a portion of the dermis with vapour pockets formed in or around the dero-epidermal junction.

Preferably, the power output device is operated to deliver discrete pulses of heat of millisecond duration.

Advantageously, the pulses have a duration in the range of from about 0.5 to about 100 milliseconds, and preferably a duration in the range of from about 4.5 to about 15.4 milliseconds.

Preferably, the flow of gas is directed through a nozzle of the instrument.

Conveniently, the power output device is operated to deliver energy in the range of from about 1 Joule to about 4 Joules, and preferably about 3.5 Joules.

In a preferred embodiment, the thermal energy source is operated to direct a jet of fluid having stored heat energy at the skin surface. Advantageously, the jet of fluid is a jet of an ionised diatomic gas.

The method provided by the invention is a cosmetic method, not a therapeutic method, being carried out to improve the appearance of the skin.

Embodiments of the invention will now be described, by way of example and with reference to the accompanying drawings, in which:

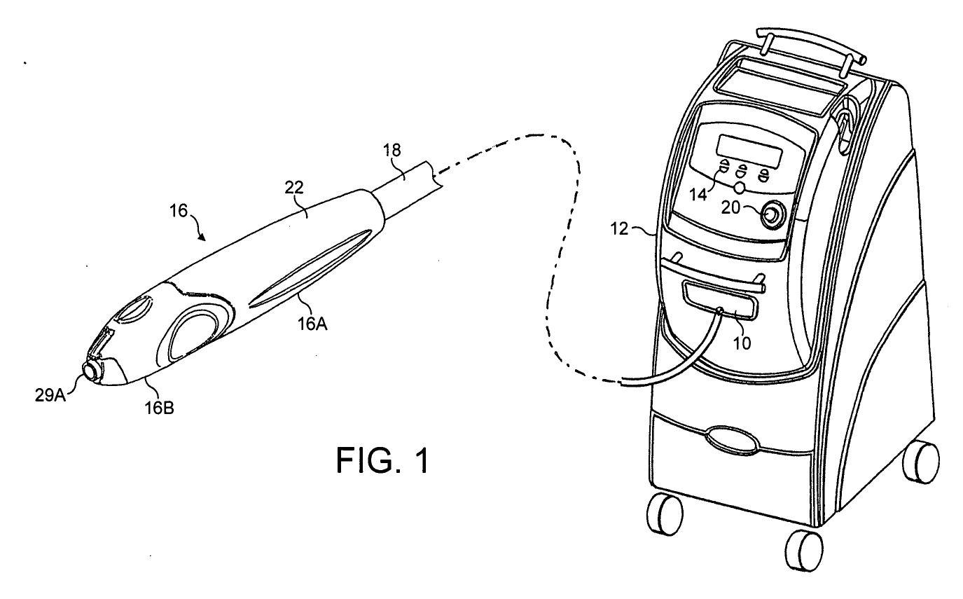

FIG. 1 is a diagrammatic view of a tissue treatment system in accordance with the invention;

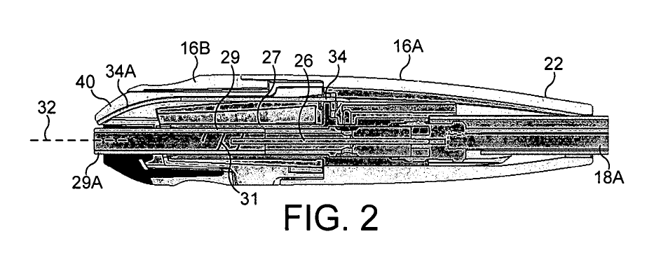

FIG. 2 is a longitudinal cross-section of a tissue treatment instrument forming part of the system of FIG. 1;

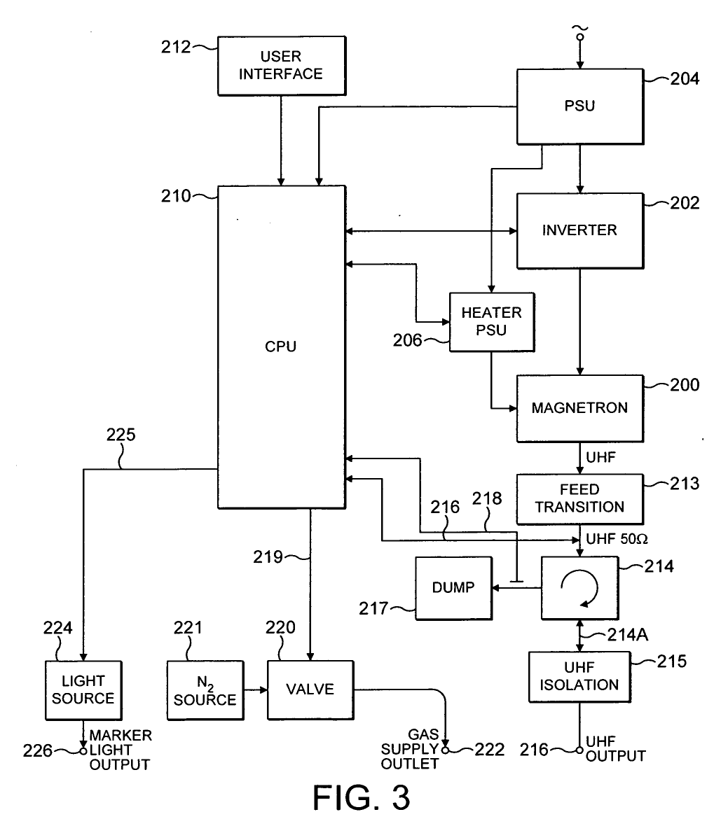

FIG. 3 is a block diagram of a radio frequency generator for use in the system of FIG. 1;

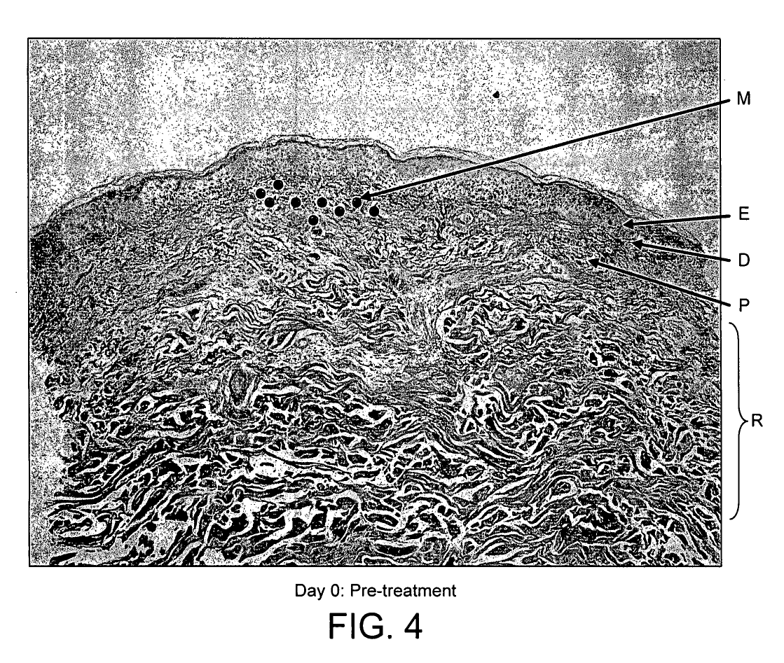

FIG. 4 shows a cross-sectional microscopic image of human forearm skin, being a typical location for tattoos, before treatment;

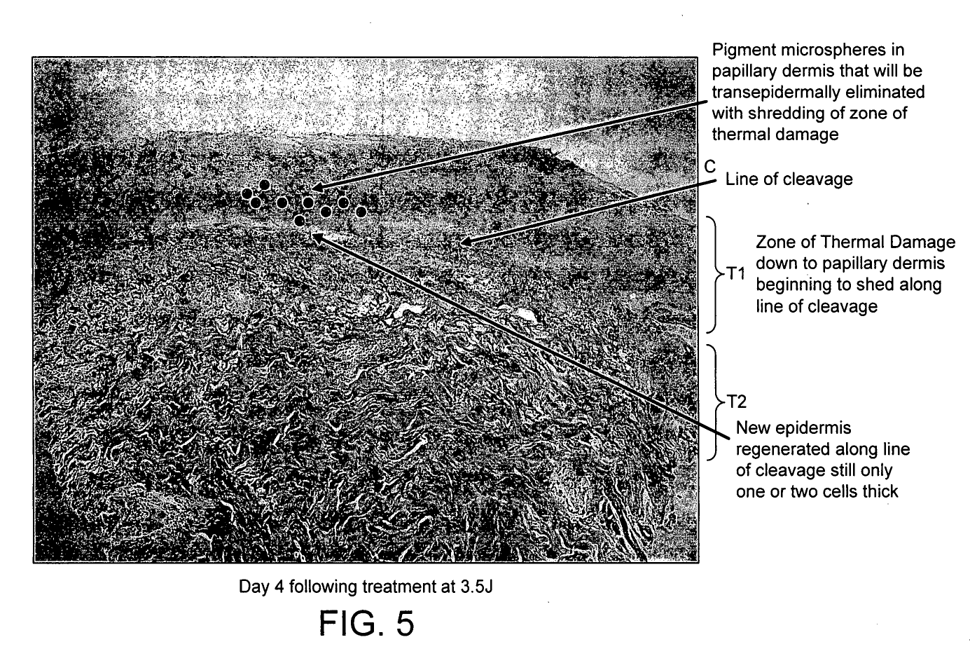

FIG. 5 shows a cross-sectional microscopic image of skin from the same subject four days following treatment;

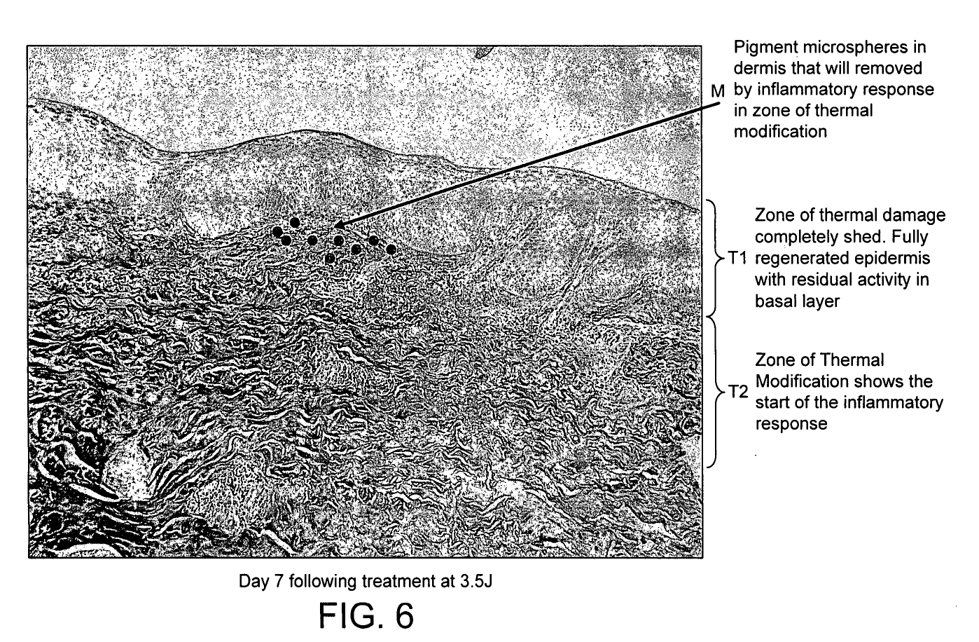

FIG. 6 shows a cross-sectional microscopic image being from the same subject seven days following treatment; and

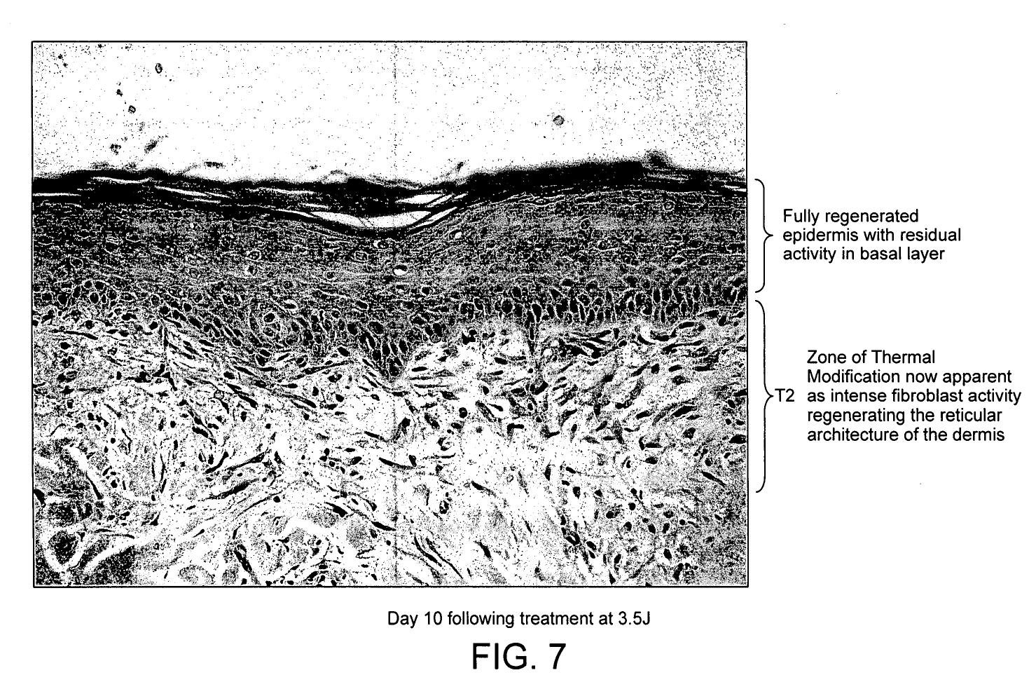

FIG. 7 shows a cross-sectional microscopic image of skin from a different subject, ten days following treatment.

Referring to FIG. 1, a tissue treatment system in accordance with the invention has a treatment power source in the form of a radio frequency (r.f) generator 10 mounted in a floor-standing generator housing 12 and having a user interface 14 for setting the generator to different energy level settings. A handheld tissue treatment instrument 16 is connected to the generator by means of a cord 18. The instrument 16 comprises a handpiece having a re-usable handpiece body 16A and a disposable nose assembly 16B.

The generator housing 12 has an instrument holder 20 for storing the instrument when not in use.

Within the cord 18 there is a coaxial cable for conveying r.f. energy from the generator 10 to the instrument 16, and a gas supply pipe for supplying nitrogen gas from a gas reservoir or source (not shown) inside the generator housing 12. The cord also contains an optical fibre line for transmitting visible light to the instrument from a light source in the generator housing. At its distal end, the cord 18 passes into the casing 22 of the handpiece body 16A

In the re-usable handpiece body 16A, the coaxial cable 18A is connected to inner and outer electrodes 26 and 27, as shown in FIG. 2. The inner electrode 26 extends longitudinally within the outer electrode 27. Between them is a heat-resistant tube 29 (preferably made of quartz) housed in the disposable instrument nose assembly 16B. When the nose assembly 16B is secured to the handpiece body 16A, the interior of the tube 29 is in communication with the gas supply pipe interior, the nose assembly 16B being received within the body 16A such that the inner electrode 26 extends axially into the tube 29 and the outer electrode 27 extends around the outside of the tube 29.

A resonator in the form of a helically wound tungsten coil 31 is located within the quartz tube 29, the coil being positioned such that, when the disposable nose assembly 16B is secured in position on the handpiece body 16A, the proximal end of the coil is adjacent the distal end of the inner electrode 26. The coil is wound such that it is adjacent and in intimate contact with the inner surface of the quartz tube 29.

In use of the instrument, nitrogen gas is fed by a supply pipe to the interior of the tube 29 where it reaches a location adjacent the distal end of the inner electrode 26. When an r.f. voltage is supplied via the coaxial cable to the electrodes 26 and 27, an intense r.f. electric field is created inside the tube 29 in the region of the distal end of the inner electrode. The field strength is aided by the helical coil 31 which is resonant at the operating frequency of the generator and, in this way, conversion of the nitrogen gas into a plasma is promoted, the plasma exiting as a jet at a nozzle 29A of the quartz tube 29. The plasma jet, centred on a treatment beam axis 32 (this axis being the axis of the tube 29), is directed onto tissue to be treated, the nozzle 29A typically being held a few millimetres from the surface of the tissue.

The handpiece 16 also contains an optical fibre light guide 34 which extends through the core 18 into the handpiece where its distal end portion 34A is bent inwardly towards the treatment axis defined by the quartz tube 29 to terminate at a distal end which defines an exit aperture adjacent the nozzle 29A. The inclination of the fibre guide at this point defines a projection axis for projecting a target marker onto the tissue surface, as will be described in more detail below.

Following repeated use of the instrument, the quartz tube 29 and its resonant coil 31 require replacement. The disposable nose assembly 16B containing these elements is easily attached and detached from the reusable part 16A of the instrument, the interface between the two components 16A, 16B of the instrument providing accurate location of the quartz tube 29 and the coil 31 with respect to the electrodes 26, 27.

Referring to FIG. 3, r.f. energy is generated in a magnetron 200. Power for the magnetron 200 is supplied in two ways, firstly as a high DC voltage for the cathode, generated by an inverter 202 supplied from a power supply unit 204 and, secondly, as a filament supply for the cathode heater from a heater power supply unit 206. Both the high voltage supply represented by the inverter 202 and the filament supply 206 are coupled to a CPU controller 210 for controlling the power output of the magnetron. A user interface 212 is coupled to the controller 210 for the purpose of setting the power output mode, amongst other functions.

The magnetron 200 operates in the high UHF band, typically at 2.475 GHz, producing an output on an output line which feeds a feed transition stage 213 for converting the waveguide magnetron output to a coaxial 50 ohms feeder, low frequency AC isolation also being provided by this stage. Thereafter, a circulator 214 provides a constant 50 ohms load impedance for the output of the feed transition stage 213. Apart from a first port coupled to the transition stage 213, the circulator 214 has a second port 214A coupled to a UHF isolation stage 215 and hence to the output terminal 216 of the generator for delivering RF power to the handheld instrument 16 (FIG. 1). Reflected power is fed from the circulator 214 to a resistive power dump 215. Forward and reflected power sensing connections 216 and 218 provide sensing signals for the controller 210.

The controller 210 also applies via line 219 a control signal for opening and closing a gas supply valve 220 so that nitrogen gas is supplied from the source 221 to a gas supply outlet 222 from where it is fed through the gas supply pipe in the cord 18 to the instrument 16 (FIG. 1), when required. A light source 224, forming part of the above-mentioned optical target marker projector, is connected to the controller 210 by a control line 225 and produces a target marker light beam at an optical marker light output 226.

The controller 210 is programmed to pulse the magnetron 200 so that, when the user presses a footswitch (not shown in the drawings), r.f. energy is delivered as a pulsed waveform to the UHF output 216, typically at a pulse repetition rate of about 4 Hz. The controller 210 also operates the valve 220 so that nitrogen gas is supplied to the handheld instrument simultaneously with the supply of r.f. energy. The light source 224 can be actuated independently of r.f. energy and nitrogen gas supply. Further details of the modes of delivery of r.f. energy are set out in U.S. Pat. No. 6,723,091, filed on 22 Feb. 2001, the disclosure of which (including the specification, the drawings and the claims) is incorporated herein by reference in its entirety.

In use, the instrument 16 is passed over the surface of tissue to be cosmetically treated, with the nozzle 29a typically being held a few millimetres from the surface of the tissue. The instrument 16 is powered to deliver pulses of 3.5 J plasma energy, each pulse producing a substantially circular treatment area 6 to 8 mm in diameter. The instrument 16 is moved, between pulses, so that adjacent treatment areas (spots) overlap by 10 to 20%.

The instrument 16 thus constitutes a thermal energy source with a low thermal time constant. The thermal time constant of an object is the product of thermal capacitance and thermal resistance, and is the time required for the temperature of the body to change by 63.2% of the difference between its initial and final temperatures when the measurements are made under zero-power conditions in a thermally stable environment. Devices which typically have low thermal time constants are those used for dynamically measuring temperature changes, the thermal time constant typically being of the order of 200 ms or less, and in micro-engineered devices this may be reduced to below 100 ms.

When the stored energy of a plasma impacts the skin, the pulse length is typically of the order of 15 ms for the transfer of energies of the order of 4 Joules, which raises the surface temperature of the skin to approximately 180° C. or 145° C. above ambient. The thermal time constant for the skin/plasma interaction will be the time taken for the surface temperature of the skin to fall by 91.6° C. Experimentally, this has been shown to occur in less than 200 ms. Once the plasma has given up its energy, it returns to the inert diatomic gas from which it was created, such that the heated skin surface is now exposed to ambient temperature as opposed to an object with a high thermal capacity, such as a hot metallic object, that will extend the thermal time constant. The larger the thermal time constant at the skin surface, the more damage and disruption will occur, as cell death is not purely correlated to temperature, but also to the time of exposure to that temperature. The plasma, therefore, has a predictable effect for a given amount of energy. Hence, it is desirable, when applying thermal energy to the skin surface, to produce a temperature elevation with a low thermal time constant.

In practice the thermal time constant should be less than 500 ms, and preferably less than 200 ms.

FIG. 4 shows the skin of a patient having a tattoo to be removed by the method of the invention, and shows the epidermis E, the DEJ J, the papillary dermis P and the reticular dermis R. Pigment microspheres M can be seen in the papillary dermis P, these microspheres constituting part of the tattoo to be removed. FIG. 5 shows that, four days following treatment, two regions T1 and T2 have been formed, T1 being an upper regional of thermal damage, and T2 being a lower region of thermal modification. The region T1 of thermal damage is a region where the temperature is sufficient to induce cellular death, and the region T2 of thermal modification is a region which is heated to a degree that denatures, but does not destroy, the dermal architecture.

FIG. 5 also shows a line of cleavage C which develops between these two regions T1 and T2 at a level consistent with the papillary dermis P. The region T1 becomes eliminated from the body, by shedding, along the line of cleavage C, once new epidermis has regenerated overlying the region T2. The region T2 then undergoes an intense inflammatory response, where denatured tissue and cellular debris is removed by inflammatory cells, and replaced by new cells and connective tissue. The new epidermis can be seen regenerating in the line of cleavage C, the new epidermis overlying the zone of thermal modification T2. As shown, pigment microspheres M above the line of cleavage C are eliminated as the zone T1 of thermal damage is shed.

As will be apparent, the depth of effect increases as the energy level and pulse width used for the treatment increases. The dermatologist carrying out the procedure will, therefore, choose the appropriate energy level and pulse width depending on the depth of the effect required. In other words, the depth of the line of cleavage C can be varied according to the energy and width of the plasma pulse. When the line of cleavage C is below the DEJ J, and in the upper papillary dermis P, then the shedding will result in the transepidermal elimination of pigment microspheres M contained therein. Pigments retained in the region T2 will be phagocytosed by the inflammatory response induced by the thermal modification. Consequently, pigment microspheres M are removed, partly by the shedding of the zone T1 of thermal damage, and partly by the inflammatory response induced in the zone T2 of thermal modification.

FIG. 5 also shows the pigment microspheres M within the upper region of thermal damage T1. At this stage, the layer of thermal damage T1 is beginning to shed along the line of cleavage C, and a new epidermis is beginning to regenerate along the line of cleavage, this regeneration being only one or two cells in thickness at this stage.

FIG. 6 shows that, seven days following treatment, the zone of thermal damage T1 has been completely shed, the zone of thermal modification T2 shows the start of what is known as an inflammatory response, and the pigment microspheres M positioned within the zone T2 of thermal modification following full regeneration of the epidermis. Thus, inflammatory response is what occurs in the zone T2 of thermal modification, the inflammation being effective to mop up the cells containing pigment microspheres M. Some residual activity in the base layer may still be occurring at this stage. Thus, the new epidermis and the DEJ have been fully regenerated with no evidence of scarring.

FIG. 7 shows that, 10 days after treatment, a more intense area of inflammatory response is formed in the zone T2 of thermal modification. This figure shows a fully regenerated epidermis with residual activity in the base layer, and the zone T2 of thermal modification is now apparent as intense fibroblast activity regenerating the reticular architecture of the dermis. As will be seen, the pigment microspheres M have been completely eliminated.

A benefit of using a diatomic plasma is that it is able to deliver a relatively large amount of energy which causes heating in a short period of time. This enables delivery in discreet pulses of millisecond duration, and is in contrast to heat conduction from a merely hot gas.

The method of the invention is particularly advantageous in that the regeneration of the epidermis and the DEJ beneath the first region T1, prior to shedding, considerably reduces or eliminates the risk of scarring.

Another advantage is that the regeneration of the dermal architecture will occur overlying any residual pigment microspheres M lying deeper in the dermis, such that the pigment colours will become more diffused. Should the diffusion be inadequate, or pigment microspheres M migrate into the newly-regenerated dermal architecture, then a second treatment can be used, once the healing response is 60 to 70% complete at about three months following treatment.

Another benefit is that oxygen is purged from the skin surface by the plasma and flow of inert gas that follows immediately following a plasma pulse. As a result, the oxidative carbonisation that often occurs at the skin surface on application of thermal energy is avoided, leaving a desiccated intact epithelium with minor structural alteration.

This minor structural alteration is nonetheless important in providing yet another benefit of the invention, as it changes the thermal characteristics of the epidermis at higher energy settings. Following a single pass of plasma over the skin surface at an energy setting greater than 2 Joules, the epidermal cells at the basal membrane are heated to a degree that produces vacuolation of the cellular contents. This produces a natural insulator limiting the absorption and depth of penetration of energy from subsequent passes. This is a beneficial safety feature that avoids the risk of excessive damage by inadvertent application of multiple passes to the same site on the skin surface.

Experiments have also shown that the insulative effect of vacuolation takes up to 20 seconds to become effective following application of energy greater than 2 Joules. If a second application is made to the same target site within 20 seconds, then the depth of each of the first and second regions is increased, such that pigments retained more deeply within the dermis may be eliminated.

The reason for using a diatomic plasma which delivers a relatively large amount of energy in a short period of time is that the irreversible clinical effects (the thermal modification and thermal damage of the tissue) occur over tissue depths that result in the desired clinical effects, whilst avoiding any undesired clinical effects. If the heating energy is delivered over too long a time, the effects of convection from the skin surface and conduction into the underlying tissue will be such that no significant temperature rise results. On the other hand, if the time is too short, then irreversible effects (such as water vaporising) at or near the skins surface will carry away otherwise useful heating energy.

To one skilled in the art, it is apparent that the above effects, and method described below, can be achieved using the delivery of heating energy to the skin that has the characteristics of a low thermal time constant, delivery in very short duration pulses (typically 0.5 to 10 milliseconds), and that does not rely on an intermediary conversion from one energy form to another, such as a chromophore in laser energy and tissue resistivity in radio frequency energy.

It will also be apparent that mechanisms other than a plasma device may be used to deliver the heating energy. In principle, any heating source, for example a material such as a hot gas, a condensing gas such as steam, a hot liquid or a hot solid that can produce in the tissue similar changes of temperature over time and tissue depth as produced by the plasma device will produce similar clinical effects. It would also be possible to use electromagnetic radiation (including light) of appropriate frequency. A further possibility would be to heat using a local exothermic chemical reaction.

In practice, it is necessary that such mechanisms are able to deliver a similar amount of energy per unit area in a similar amount of time as that described for a plasma, to achieve the required temperature. It is necessary that such a material must have the attributes of a small thermal time constant, so that the required energy can be delivered in the required amount of time. The thermal time constant is related to a particular object rather than a particular material, but is dependent also on the thermal characteristics of the material. For example, a small hot object will rapidly cool when in contact with a cooler object, yet a larger body at the same initial temperature will cool more slowly and deliver more heating energy, even though both may have the same contact area, and be made of the same material.

1. A cosmetic method of removing a tattoo from skin tissue, the method comprising the step of operating a source of thermal energy with a low thermal time constant and directing it at the surface of the skin overlying a tattoo to be removed; forming, first and second adjacent regions of thermally-modified tissue, said first region overlying said second region and being thermally modified to a greater extent than said second region; and causing tattoo pigment(s) contained in the first region to be transepidermally eliminated, and tattoo pigment(s) in the second region to be removed by an inflammatory response.

2. A cosmetic method of removing a tattoo from skin tissue using a source of thermal energy with a low thermal time constant, the method comprising the step of 15 operating the thermal energy source and directing it at the surface of the skin overlying a tattoo to be removed; forming first and second adjacent regions respectively of thermally-damaged and thermally-modified tissue; and causing tattoo pigment(s) contained in the first region to be transepidermally eliminated, and tattoo pigment(s) in the second region to be removed by an inflammatory response.

3. A method as claimed in claim 1, wherein the thermal energy source is operated for a single pass over the skin surface, the thermal energy source being arranged to have an energy setting dependent on the desired depth of effect.

4. A method as claimed in claim 1, wherein the thermal energy source is operated over at least two passes over the skin surface, the energy levels of the passes being chosen dependent on the desired depth of effect.

5. A method as claimed in claim 3, wherein the energy setting of the thermal energy source is such as to create vacuolation on the first pass.

6. A method as claimed in claim 4, wherein the energy setting of the thermal energy source is such as not to create vacuolation on the first pass, thereby enabling a second pass without removing the treated skin.

7. A method as claimed in claim 4, wherein the second pass is applied within 20 seconds of the first pass to increase the depth of effect.

8. A method as claimed in claim 1, wherein the energy setting of the thermal energy source is such as to preserve the integrity of the epidermis as a biological dressing.

9. A method as claimed in claim 1, wherein the thermal energy source is operated so that a line of cleavage occurs within the skin 2 to 5 days following treatment, the line of cleavage occurring between said first and second regions.

10. A method as claimed in claim 9, wherein the operation of the energy source is such as to form a line of cleavage from 2 to 3 cells deep.

11. A method as claimed in claim 9, wherein the operation of the thermal energy source is such that the tissue in the first region is sloughed tissue.

12. A method as claimed in claim 11, wherein the sloughed tissue is removed once a new epidermis has been substantially generated in the region of the line of cleavage.

13. A method as claimed in claim 8, wherein the tissue below the line of cleavage in said second region includes the lower epidermis, the basal membrane and the DE Junction.

14. A method as claimed in claim 13, wherein at least the thermally-modified basal 30 membrane and the DE Junction are regenerated.

15. A method as claimed in claim 8, wherein the line of cleavage forms below areas of retained tattoo pigment(s).

16. A method as claimed in claim 1, wherein the operation of the thermal energy source is such as to denature cellular elements containing tattoo pigment(s) in the second region.

17. A method as claimed in claim 1, wherein the tissue in said second region undergoes a regenerative process following regeneration of the epidermis.

18. A method as claimed in claim 17, wherein the reticular architecture of the dermis is regenerated in whole, or in part, by fibroblasts less exposed to the effects of UV radiation.

19. A method as claimed in claim 17, wherein the collagen architecture of the dermis is regenerated in whole, or in part, by fibroblasts less exposed to the effects of UV radiation.

20. A method as claimed in claim 17, wherein the elastin architecture of the dermis is regenerated in whole, or in part, by fibroblasts less exposed to the effects of UV radiation.

21. A method as claimed in claim 17, wherein the GAGS of the dermis is regenerated in whole, or in part, by fibroblasts less exposed to the effects of UV radiation.

22. A method as claimed in claim 1, wherein the healing process is such that risk of scarring and hypo pigmentation is substantially eliminated.

23. A method as claimed in claim 1, wherein the source of thermal energy is an instrument having an electrode connected to a power output device, and wherein the power output device is operated to create an electric field in the region of the electrode; a flow of gas is directed through the electric field to generate, by virtue of the interaction of the electric field with the gas, a plasma; the plasma is directed onto the tissue for a predetermined period of time; and the power transferred into the plasma from the electric field is controlled so as to desiccate at least a portion of the dermis with vapour pockets formed in dermis cells.

24. A method as claimed in claim 23, wherein the power output device is operated to deliver discrete pulses of heat of millisecond duration.

25. A method as claimed in claim 24, wherein the pulses have a duration in the range of from about 0.5 to about 100 milliseconds.

26. A method as claimed in claim 25, wherein the pulses have a duration in the range of from about 4.5 to about 15.4 milliseconds.

27. A method as claimed in claim 23, wherein the flow of gas is directed through a nozzle of the instrument.

28. A method as claimed in claim 23, wherein the power output device is operated to deliver energy in the range of from about 1 Joule to about 4 Joules.

29. A method as claimed in claim 28, wherein the device is operated to deliver energy of about 3.5 Joules.

30. A method as claimed in claim I, wherein the thermal energy source is operated to direct a jet of fluid having stored heat energy at the skin surface.

31. A method as claimed in claim 30, wherein the jet of fluid is a jet of an ionised diatomic gas.

32. A source of thermal energy with a low thermal time constant, for use in removing a tattoo from skin tissue.
